(19) United States
(12) Patent Application Publication
(10) Pub. No.: US 2007/0014823 A1
(43) Pub. Date: Jan. 18, 2007

(54) Multi phase personal care composition comprising compositions having similar rheology profile in different phases

(75) Inventor: Toshiyuki Iwata, Kobe (TW)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company

(21) Appl. No.: 11/481,512

(22) Filed: Jul. 6, 2006

Related U.S. Application Data
(60) Provisional application No. 60/698,563, filed on Jul. 12, 2005.

(51) Int. Cl. A61K 8/37; A61K 8/41

(52) U.S. Cl. 424/401

(57) ABSTRACT

Multi-phase personal care compositions comprising: (a) at least one personal care composition phase; and (b) at least one another personal care composition phase; wherein the phase (a) and said the phase (b) are visually distinct phases that are packaged in physical contact, wherein the phase (a) and the phase (b) have a yield stress of from about 1 Pa to about 100 Pa, and wherein the viscosity ratio of the phase (a) to the phase (b) is from about 1:15 to about 15:1 at shear stress over the yield stress of the phases (a) and (b) up to at least 200 Pa. By matching rheology profiles of each phase compositions in such kinetic conditions, desired patterns of multi-phase personal care compositions are easily obtained and/or maintained for a longer period of time.

### CROSS REFERENCE TO RELATED APPLICATION

This application claims the benefit of U.S. Provisional Application No. 60/698,563, filed on Jul. 12, 2005.

### FIELD OF THE INVENTION

The present invention relates to multi-phase personal care compositions comprising: (a) at least one personal care composition phase; and (b) at least one another personal care composition phase; wherein the phase (a) and said the phase (b) are visually distinct phases that are packaged in physical contact, wherein the phase (a) and the phase (b) have a yield stress of from about 1 Pa to about 100 Pa, and wherein the viscosity ratio of the phase (a) to the phase (b) is from about 1:15 to about 15:1 at shear stress over the yield stress of the phases (a) and (b) up to at least 200 Pa. By matching rheology profiles of each phase compositions in such kinetic conditions, desired patterns of multi-phase personal care compositions are easily obtained and/or maintained for a longer period of time.

### BACKGROUND OF THE INVENTION

A variety of approaches have been used to condition the hair. These range from post-shampooing hair rinses, to leave-on hair conditioners, to inclusion of hair conditioning components in shampoos. Although many consumers prefer the ease and convenience of a shampoo that includes a conditioner, other consumers prefer the more conventional conditioner formulations, which are applied to hair as a separate step from shampooing, usually subsequent to shampooing. These hair conditioners are typically formulated as a single phase, thickened product, such as a gel or cream, for ease of dispensing and application to the hair.

Hair rinse conditioners have conventionally been based on the combination of a cationic surfactant, which is generally a quaternary ammonium compound such as ditallow dimethyl ammonium chloride, and fatty alcohols, such as cetyl and stearyl alcohols. This combination results in a gel matrix structure, which provides the composition with a thick, creamy rheology. However, this unique rheology of the gel matrix is extremely sensitive to additional ingredients. Addition of such ingredients results in destruction of gel matrix structure and significant reduction of the rheology.

It has been long desired to provide consumers with multiple benefits from a single product and/or provide consumers with beauty product appearance. The “multiple benefits”, for example in a hair conditioning area, include, enhanced hair conditioning, hair conditioning and styling, hair conditioning and volumizing, hair conditioning and hair shine enhancement, hair conditioning and coloring, hair conditioning and moisturization, hair conditioning and enhanced fragrance, hair conditioning and anti-dandruff, hair conditioning and UV protection, and wet hair conditioning and dry hair conditioning benefits.

One attempt at providing multiple benefits from a personal care product and/or providing an attractive product appearance, is multi-phase personal care composition having visually distinct phases. By having visually distinct phases such as multiple colors and/or transparency, the consumer can perceive visually enhanced multiple benefits from the multi-phase personal care composition. By having visually distinct phases such as multiple colors and/or transparency, consumers can also obtain an attractive product appearance from the multi-phase personal care composition, especially when the phases form, for example, specific patterns such as striping, marbling, geometries, spirals, and mixtures thereof.

When the multi-phase personal care composition forms specific patterns, the phases are usually packaged in physical contact. In such multi-phase personal care compositions wherein the phases form patterns and are packaged in physically contact, there remains a need for providing multi-phase personal care composition having such patterns stably.

It has been found by the inventors of the present invention that, in multi-phase personal care compositions wherein the phases form specific patterns and are packaged in physically contact, it is difficult to obtain desired patterns. For example, when packing the phases into the package, it is sometimes difficult to obtain desired patterns. For example, even if desired patterns are obtained, such patterns are sometimes not stable, for example, during the transportation due to vibration. It has been found by the inventors of the present invention that, especially when conditioning compositions comprise a gel matrix, it is difficult to obtain and/or maintain desired patterns.

Accordingly, the need still remains for a multi-phase personal care composition which provides multiple benefits delivered from one product and/or provides beauty product appearance, and which is easy to obtain and/or maintain desired patterns for a longer period of time. There also remains a need for a multi-phase personal care composition which provides multiple benefits delivered from one product and/or provides beauty product appearance, and which is easy to obtain and/or maintain desired patterns for a longer period of time, especially when the composition comprises a gel matrix.

### SUMMARY OF THE INVENTION

The present invention is directed to multi-phase personal care compositions comprising:

(a) at least one personal care composition phase; and

(b) at least one another personal care composition phase;

wherein the phase (a) and said the phase (b) are visually distinct phases that are packaged in physical contact, wherein the phase (a) and the phase (b) have a yield stress of from about 1 Pa to about 100 Pa, and wherein the viscosity ratio of the phase (a) to the phase (b) is from about 1:15 to about 15:1 at shear stress over the yield stress of the phases (a) and (b) up to at least 200 Pa.

The multi-phase personal care composition of the present invention provides multiple benefits delivered from one product and/or provides beauty product appearance, and which is easy to obtain and/or maintain desired patterns for a longer period of time. It has been found that, by matching rheology profiles of each phase compositions in such kinetic conditions, desired patterns of multi-phase personal care compositions are easily obtained and/or maintained for a longer period of time. The composition may optionally comprise additional components providing benefits such as conditioning, styling, coloring, volumizing, shine, health enhancement, and moisturizing.

### DETAILED DESCRIPTION OF THE INVENTION

The essential components of the personal care composition are described below. Also included is a nonexclusive description of various optional and preferred components useful in embodiments of the present invention. While the specification concludes with claims that particularly point out and distinctly claim the invention, it is believed the present invention will be better understood from the following description.

All percentages, parts and ratios are based upon the total weight of the compositions of the present invention, unless otherwise specified. All such weights as they pertain to listed ingredients are based on the active level and, therefore do not include solvents or by-products that may be included in commercially available materials, unless otherwise specified. The term “weight percent” may be denoted as “wt %” herein.

All molecular weights as used herein are weight average molecular weights expressed as grams/mole, unless otherwise specified.

The term “water soluble” as used herein, means that the component is soluble in water in the present composition. In general, the component should be soluble at about 25° C. at a concentration of about 0.1% by weight of the water solvent, preferably at about 1%, more preferably at about 5%, even more preferably at about 15%.

Herein, “comprising” means that other steps and other ingredients which do not affect the end result can be added. This term encompasses the terms “consisting of” and “consisting essentially of”.

Herein, “mixtures” is meant to include a simple combination of materials and any compounds that may result from their combination.

Multi-Phase Personal Care Compositions

The present invention relates to multi-phase personal care compositions suitable for use on mammalian hair or skin. The multi-phase personal care composition comprising: (a) at least one personal care composition phase (hereinafter Phase A and/or Phase A composition); and (b) at least one another personal care composition phase (hereinafter Phase B and/or Phase B composition). Phase A and Phase B are visually distinct phases that are packaged in physical contact.

By the term “multi-phased” or “multi-phase” as used herein, is meant that at least two phases occupy separate and distinct physical spaces inside the package in which they are stored, but are in direct contact with one another (i.e., they are not separated by a barrier and they are not emulsified). In one preferred embodiment of the present invention, the “multi-phased” personal care compositions comprising at least two phases are present within the container as a visually distinct pattern. The pattern results from the mixing or homogenization of the “multi-phased” composition. The patterns include but are not limited to the following examples: striped, marbled, rectilinear, interrupted striped, check, mottled, veined, clustered, speckled, geometric, spotted, ribbons, helical, swirl, arrayed, variegated, textured, grooved, ridged, waved, sinusoidal, spiral, twisted, curved, cycle, streaks, striated, contoured, anisotropic, laced, weave or woven, basket weave, spotted, and tessellated. Preferably the pattern is selected from the group consisting of striped, swirl, spiral, marbled and combinations thereof. In a preferred embodiment the striped pattern may be relatively uniform and even across the dimension of the package. Alternatively, the striped pattern may be uneven, i.e. wavy, or may be non-uniform in dimension. The striped pattern does not need to necessarily extend across the entire dimension of the package. The phases may be various different colors, or include particles, glitter or pearlescence.

By the term “visually distinct,” as used herein, is meant that the regions occupied by each phase can be separately seen by the human eye as distinctly separate regions in contact with one another (i.e. they are not emulsions or dispersions of particles of about 100 microns or less).

In the multi-phase personal care composition of the present invention, Phase A is present in an amount of from about 1% to about 99%, preferably from about 3% to about 97%, more preferably from about 10% to about 95%, still more preferably from about 20% to about 90% by weight of the composition. Phase B is present in an amount of from about 1% to about 95%, preferably from about 5% to about 90%, and more preferably from about 10% to about 80%, still more preferably from about 15% to about 60% by weight of the composition Although the multi-phase personal care composition of the present invention can contain other phases than Phase A and Phase B, it is preferred that the multi-phase personal care composition of the present invention consists of Phase A and Phase B. In such case, Phase A and Phase B is present in the composition at a level such that the weight ratio of Phase A to Phase B is within the range of preferably from about 99:1 to about 50:50 more preferably from about 97:3 to about 60:40 still more preferably from about 95:5 to about 65:35.

The term “personal care composition” as used herein, unless otherwise specified, refers to the compositions of the present invention, wherein the compositions are intended to include only those compositions for topical application to the hair or skin, and specifically excludes those compositions that are directed primarily to other applications such as hard surface cleansing, fabric or laundry cleansing, and similar other applications not intended primarily for topical application to the hair or skin. The personal care compositions include, for example, hair care compositions such as hair conditioning compositions, hair shampoo compositions, hair styling compositions and hair colorant compositions, and skin care compositions such as skin moisturizing compositions and skin cleansing compositions.

The compositions of the present invention preferably have a pH of from about 2 to about 8.5, more preferably from about 3 to about 7.5, even preferably from about 3.5 to about 6.5.

Rheology/Viscosity of the Composition

Generally, the viscosity of the personal care composition decreases according to the increase of shear stress. At a lower shear stress, the viscosity of the composition does not change. At a yield stress, the viscosity of the composition starts to decrease. Then, the decrease of the viscosity stops at a certain shear stress, and viscosity becomes almost constant at any higher shear stress than that shear stress. Generally, the smaller shear stress range up to the yield stress is called as “First Newtonian Plateau” in which the viscosity does not change. The larger shear stress range in which the viscosity does not change is called as “Second Newtonian Plateau”. The range from First Newtonian Plateau and Second Newtonian Plateau is called as “Power Law Region”. The viscosity in First Newtonian Plateau is called as “zero shear viscosity”.

In the present invention, Phase A and Phase B have a yield stress of from about 1 Pa to about 100 Pa, preferably from about 3 Pa to about 100 Pa, more preferably from about 10 Pa to about 100 Pa. The yield stress is measured at 25° C. by shear stress ramp measurement using AR2000 available from TA Instruments. Phase A and Phase B preferably have a zero shear viscosity of from about 10 kPa·s to about 1,000 kPa·s, more preferably from about 20 kPa·s to about 800 kPa·s, still more preferably from about 50 kPa·s to about 600 kPa·s. The zero shear viscosity is measured at 25° C. by creep method using AR2000 available from TA Instruments. The viscosity ratio of Phase A to Phase B is from about 1:15 to about 15:1, and preferably from about 1:5 to about 5:1, and more preferably from about 1:3 to about 3:1 at shear stress over yield stress of Phases A and B, up to at least 200 Pa, preferably to at least 250 Pa, more preferably up to at least a shear stress reaching to Second Newtonian Plateau of Phases A and B. The viscosities at shear stresses over yield stress of Phases A and B are measured at 25° C. by shear stress ramp measurement using AR2000 available from TA Instruments.

It is found that, difficulties in obtaining desired patterns when packing into packages are due to significant differences between rheology profiles of each phase. It has also found that difficulties in maintaining desired patterns, especially during transportation and/or rough usage such as shaking and dropping, are also due to significant differences between rheology profiles of each phase. It has been found that, by matching rheology profiles of each phase compositions in such kinetic conditions, desired patterns of multi-phase personal care compositions are easily obtained and/or maintained for a longer period of time.

Preferably, Phase A and Phase B have a density of from about 0.85 g/cm³ to about 1.15 g/cm³, more preferably from about 0.9 g/cm³ to about 1.1 g/cm³. It also preferred that the density difference between Phase A and Phase B is about 0.20 g/cm³ or less, preferably about 0.15 g/cm³ or less, more preferably about 0.10 g/cm³ or less, still more preferably about 0.05 g/cm³ or less, and even more preferably about 0.01 g/cm³ or less, in view of further improving stability under stress conditions such as vibration. The density of each phase is measured by a Pycnometer. Density is calculated in g/cm³ units.

The multi-phase personal care compositions of the present invention can contain other phases than Phase A and Phase B. When the multi-phase personal care compositions contain such additional phases, such additional phases are preferably visually distinct phases that are packaged in physical contact with at least either Phase A or Phase B. It is preferred in the present invention that such additional phases also have the above properties which are required in Phases A and B, i.e., the above specific yield stress, zero shear viscosity, viscosity ratio, and density.

Gel Matrix

In a preferred embodiment of the multi-phase personal care composition of the present invention, both Phase A and Phase B comprise a gel matrix comprising a cationic surfactant, a high melting point fatty compound, and an aqueous carrier. In more preferred embodiments, Phase B further contains a particle and a carrier fluid for the particle. In further preferred embodiments, Phase A and Phase B are as follows:

Phase A comprising:

(i) a gel matrix comprising a cationic surfactant, a high melting point fatty compound, and an aqueous carrier; and
(ii) a silicone compounds;
   wherein Phase A is substantially free of a particle having a particle size of from about 5 nm to about 5 microns; and
   Phase B comprising:
(i) a gel matrix comprising a cationic surfactant, a high melting point fatty compound, and an aqueous carrier;
(ii) a particle having a particle size of from about 5 nm to about 5 microns; and
(iii) a carrier fluid for the particle having a ClogP value of −0.5 or less;
   and wherein Phase B is substantially free of an oily compound other than the high melting point fatty compounds.

In the present invention, “substantially free of a particle” means that Phase A contains about 0.001% or less, preferably 0.0001% or less, more preferably 0% of particles. In the present invention, “substantially free of oily compounds” means that Phase B contains 1% or less, preferably 0.5% or less, more preferably 0% of oily compounds. Such oily compounds herein are any oily compounds other than the high melting point fatty compounds and any water-insoluble oily compounds which have a water-solubility of about 0.1 g or less, preferably about 0.005 g or less, more preferably 0.001 g or less, still more preferably 0.0001 g or less per 100 g water at 25° C. Such oily compounds include, for example, silicone compounds, liquid paraffins, lipids from animals, and mineral oils.

The cationic surfactants and the fatty compounds can be the same or different types and can be at the same levels or different levels in Phase A and Phase B.

Phase A and Phase B preferably comprise a gel matrix to which optional ingredients such as silicones can be added. Gel matrix comprises a cationic surfactant, a high melting fatty compound, and an aqueous carrier, and is suitable for providing various conditioning benefits such as slippery feel on wet hair and softness and moisturized feel on dry hair. In view of providing the above gel matrix, the cationic surfactant and the high melting point fatty compound are contained at a level such that the mole ratio of the cationic surfactant to the high melting point fatty compound is in the range of, preferably from about 1:1 to 1:10, more preferably from about 1:2 to 1:6.

Preferably, Phase A and Phase B comprises by weight, from about 60% to about 99%, preferably from about 70% to about 95%, and more preferably from about 80% to about 95% of a gel matrix.

The composition containing the above amount of gel matrix is typically characterized by a viscosity of from about 5,000 cps to about 40,000 cps, preferably from about 10,000 cps to about 30,000 cps, and more preferably from about 12,000 cps to about 28,000 cps, as measured at 25° C., by means of a Brookfield Viscometer at shear rate of 1.0 rpm. Although the composition of the present invention can contain a thickening polymer, the composition of the present invention can have the above viscosity without the presence of any thickening polymer.

The existence of a gel matrix can be detected by differential scanning calorimetry (hereinafter referred to as “DSC”) measurement of the composition. A profile chart obtained by DSC measurement describes chemical and physical changes of the scanned sample that involve an enthalpy change or energy gradient when the temperature of the sample is fluctuated. As such, the phase behavior and interaction among components of hair conditioning compositions of the present invention may be understood by their DSC profiles. DSC measurement of compositions of the present invention may be conducted by any suitable instrument available. For example, DSC measurement may be suitably conducted by Seiko DSC 6000 instrument available from Seiko Instruments Inc. In a typical measurement procedure, a sample is prepared by sealing an appropriate amount of the composition into a container made for DSC measurement and sealed. The weight of the sample is recorded. A blank sample i.e.; an unsealed sample of the same container is also prepared. The sample and blank sample are placed inside the instrument, and run under a measurement condition of from about −50° C. to about 130° C. at a heating rate of from about 1° C./minute to about 10° C./minute. The area of the peaks as identified are calculated and divided by the weight of the sample to obtain the enthalpy change in mJ/mg. The position of the peaks is identified by the peak top position. In a preferred composition having a higher amount of gel matrix, the DSC profile shows a formation peak of larger than about 3 mJ/mg, more preferably from about 6 mJ/mg to about 10 mJ/mg. The DSC profile of a preferred composition shows a single peak having a peak top temperature of from about 55° C. to about 75° C., preferably from about 67° C. to about 73° C. The DSC profile of the preferred composition shows no peaks larger than 3 mJ/mg, more preferably no peaks larger than 2.5 mJ/mg, still more preferably no peaks larger than 2 mJ/mg at a temperature of from 40° C. to 55° C., as the peaks showing at a temperature of from 40° C. to 55° C. mean the existence of high melting fatty compounds and/or cationic surfactants which are not incorporated into the gel matrix. It is believed that a composition formed predominantly with such a gel matrix shows a relatively stable phase behavior during the temperature range of from about 40° C. to about 55° C.

Preferably, when Phase A and Phase B comprise a gel matrix, Phase A and Phase B are substantially free of anionic surfactants and anionic polymers in view of stability of the gel matrix. In the present invention, “substantially free of anionic surfactants and anionic polymers” means that the composition contains 1% or less, preferably 0.5% or less, more preferably totally 0% of total of anionic surfactants and anionic polymers.

Cationic Surfactant

The cationic surfactant can be included in Phase A and Phase B compositions at a level by weight of preferably from about 0.1% to about 10%, more preferably from about 1% to about 8%, still more preferably from about 2% to about 5%.

A variety of cationic surfactants including mono- and di-alkyl chain cationic surfactants can be used in the compositions of the present invention as described below. Among them, preferred are mono-alkyl chain cationic surfactants such as mono-alkyl chain quaternary ammonium salts. The mono-alkyl chain quaternary ammonium salts useful herein are those having mono-long alkyl chain which has from 12 to 22 carbon atoms, preferably from 16 to 22 carbon atoms. Highly preferred mono-alkyl chain quaternary ammonium salts are, for example, cetyl trimethyl ammonium chloride, stearyl trimethyl ammonium chloride, behenyl trimethyl ammonium chloride. Although the mono-alkyl chain cationic surfactants are preferred, other cationic surfactants such as di-alkyl chain cationic surfactants may also be used alone, or in combination with the mono-alkyl chain cationic surfactants and/or nonionic surfactants.

Cationic surfactants useful herein include, for example, those corresponding to the general formula (I): 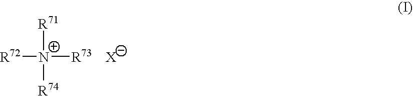
wherein at least one of R⁷¹, R⁷², R⁷³ and R⁷⁴ is selected from an aliphatic group of from 8 to 30 carbon atoms or an aromatic, alkoxy, polyoxyalkylene, alkylamido, hydroxyalkyl, aryl or alkylaryl group having up to about 22 carbon atoms, the remainder of R⁷¹, R⁷², R⁷³ and R⁷⁴ are independently selected from an aliphatic group of from 1 to about 22 carbon atoms or an aromatic, alkoxy, polyoxyalkylene, alkylamido, hydroxyalkyl, aryl or alkylaryl group having up to about 22 carbon atoms; and X is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The aliphatic groups can contain, in addition to carbon and hydrogen atoms, ether linkages, and other groups such as amino groups. The longer chain aliphatic groups, e.g., those of about 12 carbons, or higher, can be saturated or unsaturated. Preferred is when R⁷¹, R⁷², R⁷³ and R⁷⁴ are independently selected from C₁ to about C₂₂ alkyl.

Among the cationic surfactants of general formula (I), preferred are those containing in the molecule at least one alkyl chain having at least 16 carbons. Nonlimiting examples of such preferred cationic surfactants include: behenyl trimethyl ammonium chloride available, for example, with tradename Genamine KDMP from Clariant, with tradename INCROQUAT TMC-80 from Croda, and with tradename ECONOL TM22 from Sanyo Kasei; cetyl trimethyl ammonium chloride available, for example, with tradename CTAC 30KC from KCl, and with tradename CA-2350 from Nikko Chemicals; stearyl trimethyl ammonium chloride available, for example, with tradename Genamine STACP from Clariant; olealkonium chloride available, for example, with tradename Incroquat O-50 from Croda; hydrogenated tallow alkyl trimethyl ammonium chloride, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

Also preferred are hydrophilically substituted cationic surfactants in which at least one of the substituents contain one or more aromatic, ether, ester, amido, or amino moieties present as substituents or as linkages in the radical chain, wherein at least one of the R⁷¹-R⁷⁴ radicals contain one or more hydrophilic moieties selected from alkoxy (preferably C₁-C₃ alkoxy), polyoxyalkylene (preferably C₁-C₃ polyoxyalkylene), alkylamido, hydroxyalkyl, alkylester, and combinations thereof. Preferably, the hydrophilically substituted cationic conditioning surfactant contains from 2 to about 10 nonionic hydrophile moieties located within the above stated ranges. Highly preferred hydrophilically substituted cationic surfactants include dialkylamido ethyl hydroxyethylmonium salt, dialkylamidoethyl dimonium salt, dialkyloyl ethyl hydroxyethylmonium salt, dialkyloyl ethyldimonium salt, and mixtures thereof; for example, commercially available under the following tradenames; VARISOFT 110, VARISOFT 222, VARIQUAT K1215 and VARIQUAT 638 from Witco Chemical, MACKPRO KLP, MACKPRO WLW, MACKPRO MLP, MACKPRO NSP, MACKPRO NLW, MACKPRO WWP, MACKPRO NLP, MACKPRO SLP from McIntyre, ETHOQUAD 18/25, ETHOQUAD O/12PG, ETHOQUAD C/25, ETHOQUAD S/25, and ETHODUOQUAD from Akzo, DEHYQUAT SP from Henkel, and ATLAS G265 from ICI Americas. Babassuamidopropalkonium Chloride available from Croda under the tradename Incroquat BA-85 is also preferably used in the composition.

Amines are suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid. The amines herein are preferably partially neutralized with any of the acids at a molar ratio of the amine to the acid of from about 1:0.3 to about 1:2, more preferably from about 1:0.4 to about 1:1.

High Melting Point Fatty Compound

The high melting point fatty compound can be included in Phase A and Phase B compositions at a level of from about 2.5% to about 15%, preferably from about 4% to about 10%, more preferably from about 5% to about 8% by weight of the compositions.

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about 22 carbon atoms. These fatty alcohols are saturated and can be straight or branched chain alcohols. Preferred fatty alcohols include, for example, cetyl alcohol, stearyl alcohol, behenyl alcohol, and mixtures thereof.

Commercially available high melting point fatty compounds useful herein include: cetyl alcohol, stearyl alcohol, and behenyl alcohol having tradenames KONOL series available from Shin Nihon Rika (Osaka, Japan), and NAA series available from NOF (Tokyo, Japan); pure behenyl alcohol having tradename 1-DOCOSANOL available from WAKO (Osaka, Japan).

Aqueous Carrier

The Phase A and Phase B compositions of the present invention preferably comprise an aqueous carrier. The level and species of the carrier are selected according to the compatibility with other components, and other desired characteristic of the product.

The carrier useful in the present invention includes water and water solutions of lower alkyl alcohols and polyhydric alcohols. The lower alkyl alcohols useful herein are monohydric alcohols having 1 to 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product. Generally, the compositions of the present invention comprise from about 20% to about 99%, preferably from about 30% to about 95%, and more preferably from about 80% to about 95% water.

Particle

Phase A and Phase B compositions of the present invention can contain particles for providing visually distinct phases, beauty appearance and/or other benefits. In preferred embodiments, Phase B contains particles, while Phase A is substantially free of particles especially when Phase A contains an oily compound such as silicone compound. The particles can be contained in the composition at a level by weight of preferably from about 0.001% to about 10%, more preferably from about 0.005% to about 7%, still more preferably from about 0.005% to about 5%. Among the particles, pigments can be contained in the composition at a level by weight of preferably from about 0.001% to about 0.5%, more preferably from about 0.005% to about 0.2%, still more preferably from about 0.005% to about 0.1%.

The particles useful herein are water-insoluble, and those having a particle size of from about 5 nm to about 5 μm, preferably from about 5 nm to about 2 μm, more preferably from about 5 nm to about 1 μm. The particles useful herein are preferably those having a density of 0.8 g/cm³ or higher, more preferably 0.9 g/cm³ or higher.

The particles useful herein can be organic or inorganic. Organic particles include, for example, polymeric particles and organic pigments including lakes. Inorganic particles include, for example, inorganic anti-dandruff agents, inorganic UV protecting agents, inorganic fillers, and inorganic pigments. Among the above particles, preferred are organic pigments and inorganic particles.

Such organic pigments include, for example, D&C Red 30 Al lake, FD&C Blue 1 lake, FD&C Yellow 5 lake, D&C Red 30 Talc Lake, D&C Red 7 Calcium Lake, D&C Red 34 Calcium Lake, Red 30 Low Iron, D&C Red 27 Al lake, D&C Red 28 Lake, D&C Yellow 6 Lake, D&C Yellow 5 Zr Lake, intercalated FD&C Blue 1, intercalated FD&C Yellow 6, intercalated D&C Yellow 10 and intercalated D&C Red 6 wherein the intercalated organic pigments are those intercalated in the layered double hydrotalcite, and mixtures thereof. Preferred are D&C Red 30 Al lake, D&C Red 30 Talk Lake, D&C Red 27 Al lake, and intercalated FD&C Blue 1, intercalated FD&C Yellow 6, intercalated D&C Yellow 10 and intercalated D&C Red 6 wherein the intercalated organic pigments are those intercalated in the layered double hydrotalcites which have the chemical formula: [Zn₁₋ₓAlₓ(OH)₂]^{x+}(Aⁿ⁻)_{x/n}.yH₂O wherein Aⁿ⁻ represents an anionic dye molecule, and x=[Al]/([Zn]+[Al]). Such intercalated organic pigments are available from Daito Kasei Kogyo Co., Ltd.

Such inorganic particles include, for example, inorganic anti-dandruff agents such as zinc pyrrithione and zinc oxide, inorganic UV protecting agents such as zinc oxide and titanium dioxide, inorganic fillers such as calcium carbonate, barium sulfate, and calcium sulfate, and inorganic pigments.

Inorganic pigments are iron oxide, ferric ferrocyanide, chromium oxide, hydrated chromium oxide, manganese violet, ultramarine, titanium dioxide, zinc oxide, carbon black, natural mica, synthetic mica, graphite, talc, kaolin, alumina flake, bismuth oxychloride, silica flake, glass flake, ceramics, titanium dioxide, bentonite, CaSO₄, CaCO₃, BaSO₄, borosilicate, interference pigments, etc. and mixtures thereof.

The interference pigments of the present invention are platelet particulates. The interference pigment comprises a multi-layer structure, i.e., a particle substrate and thin films. A wide variety of particle substrates are useful herein. Nonlimiting examples of the particle substrates are natural mica, synthetic mica, graphite, talc, kaolin, alumina flake, bismuth oxychloride, silica flake, glass flake, ceramics, titanium dioxide, bentonite, CaSO₄, CaCO₃, BaSO₄, borosilicate and mixtures thereof, preferably mica, silica and alumina flakes. A layer of thin film or a multiple layer of thin films are coated on the surface of a substrate described above. A wide variety of thin films are useful herein. Nonlimiting examples of the thin films are TiO₂, Fe₂O₃, SnO₂, Cr₂O₃, ZnO, ZnS, ZnO, SnO, ZrO₂, CaF₂, Al₂O₃, BiOCl, and mixtures thereof or in the form of separate layers, preferably TiO₂, Fe₂O₃, Cr₂O₃, and SnO₂. Nonlimiting examples of the interference pigments useful herein include those supplied by Persperse, Inc. under the trade name PRESTIGE®, FLONAC®; supplied by EMD Chemicals, Inc. under the trade name TIMIRON®, COLORONA®, DICHRONA® and XIRONA®; and supplied by Engelhard Co. under the trade name FLAMENCO®, TIMICA®, DUOCHROME®. The interference pigment surface is either hydrophobic or has been hydrophobically modified. Nonlimiting examples of the hydrophobic surface treatment useful herein include silicones, acrylate silicone copolymers, acrylate polymers, alkyl silane, isopropyl titanium triisostearate, sodium stearate, magnesium myristate, perfluoroalcohol phosphate, perfluoropolymethyl isopropyl ether, lecithin, carnauba wax, polyethylene, chitosan, lauroyl lysine, plant lipid extracts and mixtures thereof, preferably, silicones, silanes and stearates. Surface treatment houses include US Cosmetics, KOBO Products Inc., and Cardre Inc.

Carrier Fluid for Particles

When the above particles are included in the composition of the present invention, it is preferred to use carrier fluid for the particles to disperse evenly without aggregation or agglomeration. Carrier fluid can be contained in the composition at a level such that the weight ratio of the carrier fluid to the particles is within the range of preferably from about 95:5 to about 20:80, more preferably from about 90:10 to about 40:60, still more preferably from about 80:20 to about 60:40.

Aggregations and/or agglomerations can be seen when using small particles, especially when using particles having a high density and/or electrostatic charge, more especially when using organic pigments and inorganic particles including inorganic pigments. It is preferred to prevent such aggregations and/or agglomerations in view of beauty appearance and/or usage feel, thus, it is preferred to use carrier fluids for dispersing the particles. However, as described above, the rheology of the gel matrix is extremely sensitive to additional ingredients, and the viscosity under kinetic conditions of the gel matrix is easily reduced significantly. It has been found by the inventors of the present invention, by the use of specific carrier fluids, the particles and the carrier fluids can be incorporated into the gel matrix without significant reduction of viscosity of gel matrix.

Solvents useful herein are preferably those having a ClogP value of −0.5 or less, more preferably −1.0 or less, still more preferably −1.5 or less. Such solvents useful herein include, for example, diglycerine (ClogP value=−2.955), sorbitol (ClogP=−2.046), glycerin (ClogP value=−1.538), ethanediol (ClogP value=−1.369), diethyleneglycol (ClogP value=−1.305), 1,4-butanediol (ClogP value=−1.164), propylene glycol (ClogP value=−1.037), 1,4-pentanediol (ClogP value=−0.855), 1,3-butanediol (ClogP value=−0.728), dipropyleneglycol (ClogP value=−0.662), 1,5-pentanediol (ClogP value=−0.635). Among the solvents, preferred are diglycerine, glycerin, ethanediol, diethyleneglycol, 1,4-butanediol, propylene glycol, and mixtures thereof. More preferred are diglycerine, glycerin, and mixtures thereof.

ClogP is the calculated log(partition coefficient in oil and water), calculated by the CLOGP© program by Pomona College and BioByte, Inc. of Claremont, Calif.

Silicone Compound

Phase A and Phase B can contain a silicone compounds in view of providing conditioning benefits especially smoothness and softness. However, in the present invention, it is preferred to not contain a silicone compounds together with the above particles in view of avoiding particles buildup on the surface of manufacturing equipments. Thus, in preferred embodiments, Phase A contains a silicone compound, while Phase B is substantially free of a silicone compound especially when Phase B contains the above particles.

The silicone compounds can be used at levels by weight of the composition of preferably from about 0.1% to about 20%, more preferably from about 0.5% to about 10%, still more preferably from about 1% to about 8%.

The silicone compounds useful herein, as a single compound, as a blend or mixture of at least two silicone compounds, or as a blend or mixture of at least one silicone compound and at least one solvent, have a viscosity of preferably from about 1,000 to about 2,000,000 mPa·s at 25° C.

The viscosity can be measured by means of a glass capillary viscometer as set forth in Dow Corning Corporate Test Method CTM0004, Jul. 20, 1970. Suitable silicone fluids include polyalkyl siloxanes, polyaryl siloxanes, polyalkylaryl siloxanes, polyether siloxane copolymers, amino substituted silicones, quaternized silicones, and mixtures thereof. Other nonvolatile silicone compounds having conditioning properties can also be used.

Preferably, the silicone compounds have an average particle size of from about 1 microns to about 50 microns, in the composition.

The silicone compounds useful herein include polyalkyl or polyaryl siloxanes with the following structure: 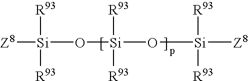
wherein R⁹³ is alkyl or aryl, and p is an integer from about 7 to about 8,000. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the hair, is compatible with the other components of the composition, is chemically stable under normal use and storage conditions, and is capable of being deposited on and conditions the hair. Suitable Z⁸ groups include hydroxy, methyl, methoxy, ethoxy, propoxy, and aryloxy. The two R⁹³ groups on the silicon atom may represent the same group or different groups. Preferably, the two R⁹³ groups represent the same group. Suitable R⁹³ groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their Viscasil® and TSF 451 series, and from Dow Corning in their Dow Corning SH200 series.

The above polyalkylsiloxanes are available, for example, as a mixture with silicone compounds having a lower viscosity. Such mixtures have a viscosity of preferably from about 1,000 mPa·s to about 100,000 mPa·s, more preferably from about 5,000 mPa·s to about 50,000 mPa·s. Such mixtures preferably comprise: (i) a first silicone having a viscosity of from about 100,000 mPa·s to about 30,000,000 mPa·s at 25° C., preferably from about 100,000 mPa·s to about 20,000,000 mPa·s; and (ii) a second silicone having a viscosity of from about 5 mPa·s to about 10,000 mPa·s at 25° C., preferably from about 5 mPa·s to about 5,000 mPa·s. Such mixtures useful herein include, for example, a blend of dimethicone having a viscosity of 18,000,000 mPa·s and dimethicone having a viscosity of 200 mPa·s available from GE Toshiba, and a blend of dimethicone having a viscosity of 18,000,000 mPa·s and cyclopentasiloxane available from GE Toshiba.

The silicone compounds useful herein also include a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. The silicone gums are available, for example, as a mixture with silicone compounds having a lower viscosity. Such mixtures useful herein include, for example, Gum/Cyclomethicone blend available from Shin-Etsu.

The silicone compounds that can be used include, for example, a polypropylene oxide modified polydimethylsiloxane although ethylene oxide or mixtures of ethylene oxide and propylene oxide can also be used. The ethylene oxide and polypropylene oxide level should be sufficiently low so as not to interfere with the dispersibility characteristics of the silicone. These materials are also known as dimethicone copolyols.

Silicone compounds useful herein also include amino substituted materials. Preferred aminosilicones include, for example, those which conform to the general formula (I):
(R₁)ₐG₃₋ₐ-Si—(—OSiG₂)ₙ—(—OSiG_{b}(R₁)_{2-b})ₘ—O—SiG₃₋ₐ(R₁)ₐ
wherein G is hydrogen, phenyl, hydroxy, or C₁-C₈ alkyl, preferably methyl; a is 0 or an integer having a value from 1 to 3, preferably 1; b is 0, 1 or 2, preferably 1; n is a number from 0 to 1,999; m is an integer from 0 to 1,999; the sum of n and m is a number from 1 to 2,000; a and m are not both 0; R₁ is a monovalent radical conforming to the general formula CqH_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical, preferably an alkyl radical from about C₁ to about C₂₀; A⁻ is a halide ion.

Highly preferred amino silicones are those corresponding to formula (I) wherein m=0, a=1, q=3, G=methyl, n is preferably from about 1500 to about 1700, more preferably 1600; and L is —N(CH₃)₂. Such highly preferred amino silicones can be called as terminal aminosilicones, as one or both ends of the silicone chain are terminated by nitrogen containing group.

The above aminosilicones, when incorporated into the composition, can be mixed with solvent having a lower viscosity. Such solvents include, for example, polar or non-polar, volatile or non-volatile oils. Such oils include, for example, silicone oils, hydrocarbons, and esters. Among such a variety of solvents, preferred are those selected from the group consisting of non-polar, volatile hydrocarbons, volatile cyclic silicones, non-volatile linear silicones, and mixtures thereof. The non-volatile linear silicones useful herein are those having a viscosity of from about 1 to about 20,000 centistokes, preferably from about 20 to about 10,000 centistokes at 25° C. Among the preferred solvents, highly preferred are non-polar, volatile hydrocarbons, especially non-polar, volatile isoparaffins, in view of reducing the viscosity of the aminosilicones and providing improved hair conditioning benefits such as reduced friction on dry hair. Such mixtures have a viscosity of preferably from about 1,000 mPa·s to about 100,000 mPa·s, more preferably from about 5,000 mPa·s to about 50,000 mPa·s.

Other suitable alkylamino substituted silicone compounds include those represented by the following structure: 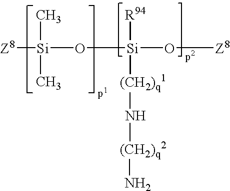
wherein R⁹⁴ is H, CH₃ or OH; p¹ and p² are integers of 1 or above, and wherein sum of p¹ and p² is from 650 to 1,500; q¹ and q² are integers of from 1 to 10. Z⁸ represents groups which block the ends of the silicone chains. Suitable Z⁸ groups include hydroxy, methyl, methoxy, ethoxy, propoxy, and aryloxy. Highly preferred are those known as “amodimethicone”. Commercially available amodimethicones useful herein include, for example, BY16-872 available from Dow Corning.

Other amino substituted silicone polymers which can be used are represented by the formula: 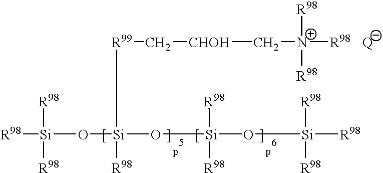
wherein R⁹⁸ denotes a monovalent hydrocarbon radical having from 1 to 18 carbon atoms, preferably an alkyl or alkenyl radical such as methyl; R⁹⁹ denotes a hydrocarbon radical, preferably a C₁-C₁₈ alkylene radical or a C₁-C₁₈, and more preferably C₁-C₈, alkyleneoxy radical; Q⁻ is a halide ion, preferably chloride; p⁵ denotes an average statistical value from 2 to 20, preferably from 2 to 8; p⁶ denotes an average statistical value from 20 to 200, and preferably from 20 to 50.

The silicone compounds may further be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and mixtures thereof.

Additional Components

The compositions of the present invention may comprise additional components. The additional components may be found in Phase A, Phase B, other phases if included, and/or all of these phases.

Humectants

A suitable benefit agent is one or more humectants. A variety of humectants can be employed and can be present at a level of from about 0.1% to about 50%, preferably from about 0.5% to about 35%, and more preferably from about 2% to about 20% by weight of a non-volatile, organic material having a solubility of at least 5 parts in 10 parts water. A preferred water soluble, organic material is selected from the group consisting of guanidine; sugars such as sorbitol and sucrose; starches; sugar and starch derivatives (e.g. alkoxylated glucose); panthenol (including D-, L-, and the D,L-forms); pyrrolidone carboxylic acid; hyaluronic acid; lactamide monoethanolamine; acetamide monoethanolamine; urea; and ethanol amines of the general structure (HOCH₂CH₂)xNHy where x=1-3; y=0-2, and x+y=3, and mixtures thereof.

Water Soluble Nonionic Polymers

The compositions of the present invention may comprise from about 0.1% to about 10%, more preferably from about 0.2% to about 5%, and even more preferably from about 0.5% to about 3% by weight of a water soluble nonionic polymer.

The polymers of the present invention are characterized by the general formula: 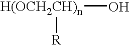
wherein R is selected from the group consisting of H, methyl, and mixtures thereof. When R is H, these materials are polymers of ethylene oxide, which are also known as polyethylene oxides, polyoxyethylenes, and polyethylene glycols. When R is methyl, these materials are polymers of propylene oxide, which are also known as polypropylene oxides, polyoxypropylenes, and polypropylene glycols. When R is methyl, it is also understood that various positional isomers of the resulting polymers can exist. In the above structure, n has an average value of from about 2,000 to about 14,000, preferably from about 5,000 to about 9,000, more preferably from about 6,000 to about 8,000.

Polyethylene glycol polymers useful herein that are especially preferred are PEG-2M wherein R equals H and n has an average value of about 2,000 (PEG 2-M is also known as Polyox WSR® N-10 from Union Carbide and as PEG-2,000); PEG-5M wherein R equals H and n has an average value of about 5,000 (PEG 5-M is also known as Polyox WSR® N-35 and Polyox WSR® N-80, both from Union Carbide and as PEG-5,000 and Polyethylene Glycol 300,000); PEG-7M wherein R equals H and n has an average value of about 7,000 (PEG 7-M is also known as Polyox WSR® N-750 from Union Carbide); PEG-9M wherein R equals H and n has an average value of about 9,000 (PEG 9-M is also known as Polyox WSR® N-3333 from Union Carbide); and PEG-14 M wherein R equals H and n has an average value of about 14,000 (PEG 14-M is also known as Polyox WSR® N-3000 from Union Carbide.) Other useful polymers include the polypropylene glycols and mixed polyethylene/polypropylene glycols.

Cationic Polymer Conditioning Agent

Cationic conditioning polymers useful herein are those having an average molecular weight of at least about 5,000, typically from about 10,000 to about 10 million, preferably from about 100,000 to about 2 million. Cationic conditioning polymers can be used in the composition of the present invention at a level by weight of preferably from about 0.05% to about 2%, more preferably from about 0.1% to about 0.5%. Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. Other suitable spacer monomers include vinyl esters, vinyl alcohol (made by hydrolysis of polyvinyl acetate), maleic anhydride, propylene glycol, and ethylene glycol. Other suitable cationic polymers useful herein include, for example, cationic celluloses, cationic starches, and cationic guar gums.

Low Melting Point Oil

Low melting point oils useful herein are those having a melting point of less than 25° C. The low melting point oil useful herein is selected from the group consisting of: hydrocarbon having from 10 to about 40 carbon atoms; unsaturated fatty alcohols having from about 10 to about 30 carbon atoms such as oleyl alcohol; unsaturated fatty acids having from about 10 to about 30 carbon atoms; fatty acid derivatives; fatty alcohol derivatives; ester oils such as pentaerythritol ester oils, trimethylol ester oils, citrate ester oils, and glyceryl ester oils; poly α-olefin oils; and mixtures thereof. Preferred low melting point oils herein are selected from the group consisting of: ester oils such as pentaerythritol ester oils, trimethylol ester oils, citrate ester oils, and glyceryl ester oils; poly α-olefin oils; and mixtures thereof,

Particularly useful pentaerythritol ester oils and trimethylol ester oils herein include pentaerythritol tetraisostearate, pentaerythritol tetraoleate, trimethylolpropane triisostearate, trimethylolpropane trioleate, and mixtures thereof. Such compounds are available from Kokyo Alcohol with tradenames KAKPTI, KAKTTI, and Shin-nihon Rika with tradenames PTO, ENUJERUBU TP3SO.

Particularly useful citrate ester oils herein include triisocetyl citrate with tradename CITMOL 316 available from Bemel, triisostearyl citrate with tradename PELEMOL TISC available from Phoenix, and trioctyldodecyl citrate with tradename CITMOL 320 available from Bemel.

Particularly useful glyceryl ester oils herein include triisostearin with tradename SUN ESPOL G-318 available from Taiyo Kagaku, triolein with tradename CITHROL GTO available from Croda Surfactants Ltd., trilinolein with tradename EFADERMA-F available from Vevy, or tradename EFA-GLYCERIDES from Brooks.

Particularly useful poly α-olefin oils herein include polydecenes with tradenames PURESYN 6 having a number average molecular weight of about 500 and PURESYN 100 having a number average molecular weight of about 3000 and PURESYN 300 having a number average molecular weight of about 6000 available from Exxon Mobil Co.

Other Additional Ingredients

The compositions herein can contain a variety of other optional components suitable for rendering such compositions more cosmetically or aesthetically acceptable or to provide them with additional usage benefits. Optional ingredients may be found in either the conditioning phase or the benefit phase. Such conventional optional ingredients are well-known to those skilled in the art.

A wide variety of additional ingredients can be formulated into the present composition. These include: other conditioning agents; viscosity modifiers such as alkanolamides and methanolamides of long chain fatty acids such as cocomonoethanol amide; crystalline suspending agents; pearlescent aids such as ethylene glycol distearate; preservatives such as benzyl alcohol, methyl paraben, propyl paraben and imidazolidinyl urea; polyvinyl alcohol; ethyl alcohol; pH adjusting agents, such as citric acid, sodium citrate, succinic acid, phosphoric acid, sodium hydroxide, sodium carbonate; salts, in general, such as potassium acetate and sodium chloride; water-soluble coloring agents, such as any of the FD&C or D&C dyes; hair coloring agents/dyes such as anthroquinone, azo, nitro, basic, triarylmethane, or disperse dyes, or any combinations thereof; hair styling polymers such as polyquaternium-55 (Styleze W-10 or W-20 from ISP (International Specialty Products)), polyquaternium-68 (Luviquat Supreme from BASF); hair oxidizing (bleaching) agents, such as hydrogen peroxide, perborate and persulfate salts; hair reducing agents, such as the thioglycolates; perfumes; sequestering agents, such as disodium ethylenediamine tetra-acetate; and polymer plasticizing agents, such as glycerin, disobutyl adipate, butyl stearate, and propylene glycol. Other non limiting examples of these optional ingredients include vitamins and derivatives thereof (e.g., ascorbic acid, vitamin E, tocopheryl acetate, and the like); sunscreens; thickening agents (e.g., polyol alkoxy ester, available as Crothix from Croda); preservatives for maintaining the anti microbial integrity of the conditioning compositions; anti-acne medicaments (resorcinol, salicylic acid, and the like); antioxidants; skin soothing and healing agents such as aloe vera extract, allantoin and the like; chelators and sequestrants; and agents suitable for aesthetic purposes such as fragrances, essential oils, skin sensates, pigments, pearlescent agents (e.g., mica and titanium dioxide), lakes, colorings, and the like (e.g., clove oil, menthol, camphor, eucalyptus oil, and eugenol).

Other optional hair and skin benefit ingredients include carboxylic acid which is hydroxylated in the α position (which compound is also referred to as an α—(alpha) hydroxyl acid) or a derivative thereof. Acid derivatives, as defined herein, are associated salts (salts with organic bases or alkali metal, for example) or lactides (obtained, for example, by autiesterification of α-hydroxy acid molecules). Examples of such compounds are, citric acid, lactic acid, methallactic acid, phenyllactic acid, malic acid, mandelic acid, glycolic acid, benzylic acid, and 2-hydroxycaprylic acid.

Additional hair and skin benefit agents include ceramides or glycoceramides. Ceramides are described in Arch. Dermatol, Vol 123, 1381-1384, 1987, or those described in French Patent FR-2,673,179; fatty acid polyesters such as, sucrose pentalaurate, sucrose tetraoleate, sucrose pentaerucate, sucrose tetraerucate, sucrose pentatallowate, sucrise triapeate, sucrose tetrapeate, sucrose pentarapeate, sucrose tristearate, and sucrose pentastearate, and mixtures thereof; polypeptides and amino acids consisting of basic amino acids, particularly arginine.

Method of Use

The personal care compositions of the present invention are used in conventional ways to provide conditioning and other benefits. Such method of use depends upon the type of composition employed but generally involves application of an effective amount of the product to the hair or skin, which may then be rinsed from the hair or skin (as in the case of hair rinses) or allowed to remain on the hair or skin (as in the case of gels, lotions, and creams). “Effective amount” means an amount sufficient enough to provide a dry combing benefit. In general, from about 1 g to about 50 g is applied to the hair on the scalp. The composition is distributed throughout the hair or skin, typically by rubbing or massaging the hair, scalp, or skin. Preferably, the composition is applied to wet or damp hair prior to drying of the hair. After such compositions are applied to the hair, the hair is dried and styled in accordance with the preference of the user. In the alternative, the composition is applied to dry hair, and the hair is then combed or styled in accordance with the preference of the user. The personal care compositions are useful in delivering conditioning benefits to hair or skin, and/or delivering hair styling benefits to hair or skin, and/or delivering hair coloring benefits to hair or skin by topically applying an effective amount of the composition onto hair or skin and removing said composition from said hair or skin by rinsing with water.

The conditioning compositions of the preferred embodiments of the present invention are especially suitable for rinse-off hair conditioner. Such compositions are preferably used by following steps:
(i) after shampooing hair, applying to the hair an effective amount of the conditioning compositions for conditioning the hair; and
(ii) then rinsing the hair.
   Method of Making

The personal care compositions of the present invention may be prepared by any known or otherwise effective technique, suitable for making and formulating the desired multi-phase product form. It is especially effective to combine toothpaste-tube filling technology with a spinning stage design. Specific non-limiting examples of such methods as they are applied to specific embodiments of the present invention are described in the following examples.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name, or otherwise defined below.

### Examples 1-4

| | Example 1 | Example 2 | Example 3 | Example 4 |
|---|---|---|---|---|
| Phase A Composition | | | | |
| Stearamidopropyl- | 2.0 | — | — | 1.0 |
| dimethylamine | | | | |
| Behenamidopropyl- | — | 2.3 | — | — |
| dimethylamine | | | | |
| L-Glutamic acid | 0.64 | 0.64 | — | — |
| Citric Acid | — | — | — | 0.13 |
| Behetrimonium Chloride | — | — | 3.381 | — |
| Ditallow dimethyl | — | — | — | 0.75 |
| ammonium chloride | | | | |
| Cetyl alcohol | 2.5 | 2.5 | 2.32 | 0.9 |
| Stearyl alcohol | 4.5 | 4.5 | 4.18 | 0.64 |
| Dimethicone blend *1 | — | — | 2.0 | — |
| Dimethicone/ | 4.2 | 4.2 | — | 4.2 |
| Cyclomethicone blend *2 | | | | |
| Hydroxyethyl cellulose | — | — | — | 0.25 |
| PEG-2M | — | — | — | 0.5 |
| Isopropanol | — | — | 0.899 | — |
| Oleyl Alcohol | — | — | — | 0.25 |
| Polysorbate-60 *3 | — | — | — | 0.25 |
| Cetearyl alcohol *3 | | | | 0.25 |
| Glyceryl Monostearate | — | — | — | 0.25 |
| Sodium hydroxide | — | — | 0.014 | — |
| Benzyl alcohol | 0.4 | 0.4 | 0.4 | 0.4 |
| EDTA | 0.1 | 0.1 | 0.1 | 0.1 |
| Kathon CG *4 | 0.033 | 0.033 | 0.033 | 0.033 |
| Panthenyl Ethyl Ether | — | 0.06 | 0.06 | — |
| Panthenol | — | 0.09 | 0.05 | — |
| Perfume | 0.6 | 0.30 | 0.25 | 0.25 |
| Deionized Water | - q.s. to 100% - | | | |
| Phase B Composition | | | | |
| Stearamidopropyl | 2.0 | — | — | 1.0 |
| dimethylamine | | | | |
| Behenamidopropyl | — | 2.3 | — | — |
| dimethylamine | | | | |
| L-Glutamic acid | 0.64 | 0.64 | — | — |
| Citric Acid | — | — | — | 0.13 |
| Behetrimonium Chloride | — | — | 3.381 | — |
| Ditallow dimethyl | — | — | — | 0.75 |
| ammonium chloride | | | | |
| Cetyl alcohol | 2.5 | 2.5 | 2.32 | 0.9 |
| Stearyl alcohol | 4.5 | 4.5 | 4.18 | 0.64 |
| Hydroxyethyl cellulose | — | — | — | 0.25 |
| PEG-2M | — | — | — | 0.5 |
| Isopropanol | — | — | 0.899 | — |
| Oleyl Alcohol | — | — | — | 0.25 |
| Polysorbate-60 *3 | — | — | — | 0.25 |
| Cetearyl alcohol *3 | | | | 0.25 |
| Glyceryl Monostearate | — | — | — | 0.25 |
| Sodium hydroxide | — | — | 0.014 | — |
| Benzyl alcohol | 0.4 | 0.4 | 0.4 | 0.4 |
| EDTA | 0.1 | 0.1 | 0.1 | 0.1 |
| Kathon CG *4 | 0.033 | 0.033 | 0.033 | 0.033 |
| Panthenyl Ethyl Ether | — | 0.06 | 0.06 | — |
| Panthenol | — | 0.09 | 0.05 | — |
| Perfume | 0.6 | 0.30 | 0.25 | 0.25 |
| Glycerin | 0.15 | 0.15 | — | — |
| Diethylene glycol | — | — | 0.3 | — |
| Diglycerine | — | — | — | 0.03 |
| D&C Red 30 Al Lake *5 | 0.05 | 0.08 | — | — |
| Manganese violet *6 | — | — | 0.1 | — |
| Titanium dioxide *7 | — | — | — | 0.02 |
| Deionized Water | - q.s. to 100% - | | | |
| Ratio Phase A/Phase B | 80/20 | 90/10 | 75/25 | 60/40 |

### Examples 5-8

| | Example 5 | Example 6 | Example 7 | Example 8 |
|---|---|---|---|---|
| Phase A Composition | | | | |
| Stearamidopropyl- | 2.0 | — | — | 2.0 |
| dimethylamine | | | | |
| Behenamidopropyl- | — | 2.3 | — | — |
| dimethylamine | | | | |
| L-Glutamic acid | 0.64 | 0.64 | — | 0.64 |
| Behetrimonium Chloride | — | — | 3.381 | — |
| Cetyl alcohol | 2.5 | 2.5 | 2.32 | 2.5 |
| Stearyl alcohol | 4.5 | 4.5 | 4.18 | 4.5 |
| Dimethicone blend *1 | — | — | 2.0 | — |
| Dimethicone/ | 4.2 | 4.2 | — | — |
| Cyclomethicone blend *2 | | | | |
| Aminosilicone *8 | — | — | — | 3.5 |
| C13-C16 Isoparaffin *9 | — | — | — | 1.5 |
| Hydroxyethyl cellulose | — | — | 0.5 | 0.75 |
| Cetyl hydroxyethyl | 0.75 | 0.25 | — | — |
| cellulose *10 | | | | |
| Sodium hydroxide | — | — | 0.014 | — |
| Benzyl alcohol | 0.4 | 0.4 | 0.4 | 0.4 |
| EDTA | 0.1 | 0.1 | 0.1 | 0.1 |
| Kathon CG *4 | 0.03 | 0.03 | 0.03 | 0.03 |
| Panthenyl Ethyl Ether | 0.225 | 0.05 | 0.225 | 0.225 |
| Panthenol | 0.050 | 0.05 | 0.050 | 0.050 |
| Perfume | 0.25 | 0.35 | 0.25 | 0.25 |
| Deionized Water | - q.s. to 100% - | | | |
| Phase B Composition | | | | |
| Stearamidopropyl- | 2.0 | — | — | 2.0 |
| dimethylamine | | | | |
| Behenamidopropyl- | — | 2.3 | — | — |
| dimethylamine | | | | |
| L-Glutamic acid | 0.64 | 0.64 | — | 0.64 |
| Behetrimonium Chloride | — | — | 3.381 | — |
| Cetyl alcohol | 2.5 | 2.5 | 2.32 | 2.5 |
| Stearyl alcohol | 4.5 | 4.5 | 4.18 | 4.5 |
| Hydroxyethyl cellulose | — | — | 0.5 | 0.75 |
| Cetyl hydroxyethyl | 0.75 | 0.25 | — | — |
| cellulose *10 | | | | |
| Sodium hydroxide | — | — | 0.014 | — |
| Benzyl alcohol | 0.4 | 0.4 | 0.4 | 0.4 |
| EDTA | 0.1 | 0.1 | 0.1 | 0.1 |
| Kathon CG *4 | 0.03 | 0.03 | 0.03 | 0.03 |
| Panthenyl Ethyl Ether | 0.225 | 0.05 | 0.225 | 0.225 |
| Panthenol | 0.050 | 0.05 | 0.050 | 0.050 |
| Perfume | 0.25 | 0.35 | 0.25 | 0.25 |
| Ethanediol | 0.1 | — | — | 0.05 |
| Diglycerine | — | 0.05 | — | — |
| 1,3-butanediol | — | 0.05 | 0.1 | 0.05 |
| D&C Red 30 Al Lake *5 | 0.03 | — | — | 0.01 |
| Iron Oxide *11 | 0.02 | 0.1 | — | — |
| Ultramarine Pink *12 | — | — | 0.05 | 0.05 |
| Deionized Water | - q.s. to 100% - | | | |
| Ratio Phase A/Phase B | 80/20 | 30/70 | 50/50 | 70/30 |
| Definitions for Components | | | | |
| *1 supplied by GE Silicones as a blend of dimethicone having a viscosity of 18,000,000 mPa · s and dimethicone having a viscosity if 200 mPa · s | | | | |
| *2 supplied by GE Silicone as a blend of dimethicone having a viscosity if 18,000,000 mPa · s and cyclopentasiloxane | | | | |
| *3 mixture sold as Polawax NF available from Croda Chemicals | | | | |
| *4 available from Rohm&Haas | | | | |
| *5 Unipure Red LC300 supplied from LCW having a particle size of 2.5 μm | | | | |
| *6 Unipure Pink LC583 supplied from LCW having a particle size of 1.8 μm | | | | |
| *7 JA-C supplied from Tayca Corp. having a particle size of 0.2 μm | | | | |
| *8 available from GE under trade name BX3083-1, having a viscosity range from 220,000-245,000 mPa · s, and having following formula (I): (R₁)ₐG₃₋ₐ-Si-(-OSiG₂)ₙ-(-OSiG_{b}(R₁)_{2-b})ₘ-O-SiG₃₋ₐ(R₁)ₐ (I) wherein G is methyl; a is an integer of 1; b is 0, 1 or 2, preferably 1; n is a number from 1500 to about 1700; m is an integer of 0; R1 is a monovalent radical conforming | | | | |
| # to the general formula CqH₂qL, wherein q is an integer of 3 and L is - N(CH₃)₂ | | | | |

*9 supplied by Nisseki as Isosol 400
*10 Polysurf 67CS available from Hercules
*11 Unipure LC380 supplied from LCW having a particle size of 0.5 μm
*12 Unipure Pink LC589 supplied from LCW having a particle size of 1.8 μm

Method of Preparation

The conditioning compositions of “Ex. 1” through “Ex. 8” as shown above can be prepared by any conventional method well known in the art. They are suitably made as follows:

Particles are added to carrier fluids with agitation in a separate vessel than the main vessel. Cationic surfactants and high melting point fatty compounds are added to water with agitation in the main vessel, and heated to a temperature above 80° C. The mixture is cooled down to about 55° C. to form a gel matrix. If included, silicone compounds, perfumes, preservatives and other remaining components are added to the gel matrix with agitation at about 55° C. The premixed particle/carrier fluid can be added at this stage, or later such before the storage tank, or just before combining two phases. In Examples containing aminosilicone and C13-C16 isoparaffin, such aminosilicone and C13-C16 isoparaffin can be pre-mixed prior to the addition to the gel matrix. Then the mixture is cooled down to room temperature.

Combine these phases by first placing the separate phases in separate storage tanks having a pump and a hose attached. Then, pump the phases in predetermined amounts into a single combining section. Next, move the phases from the combining sections into blending sections and mix the phases in the blending section such that the single resulting product exhibits a visually distinct pattern of phases and packed in physical contact. Select the pattern from the group consisting of striped, swirl, spiral, marbled, and combinations thereof. Next, pump the product that was mixed in the blending section via a hose into a single nozzle into a spinning container, and fill the container from the bottom to the top with the resulting product. Two phase products in which the phases are visually distinct and packed in physical contact are obtained. The visually distinct phases form a spiral pattern.

Such two phase products of Examples 1-8 are of the present invention, and especially useful for hair conditioners for rinse-off use. In Examples 1-8, Phase A and B compositions have a yield stress of from about 1 Pa to about 100 Pa and a zero shear viscosity of from about 10 kPa·s to about 1,000 kPa·s. In Examples 1-8, the viscosity ratio of Phase A to Phase B is from about 1:15 to about 15:1 at shear stress over the yield stress of Phases A and B up to at least 200 Pa.

The two phase products of Examples 1-8 provide at least wet hair conditioning and dry hair conditioning benefits. The two phase products of Examples 1-8 provide beauty product appearance by the visually distinct phases especially multiple colors and the pattern, and the pattern is easily obtained and maintained for a longer period of time. The two phase products of Examples 1-8 can effectively communicate consumers with multiple benefits such as wet hair conditioning and dry hair conditioning benefits, by the visually distinct phases especially multiple colors and the pattern.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A multi-phase personal care composition comprising:
(a) at least one personal care composition phase; and
(b) at least one another personal care composition phase;
wherein the phase (a) and said the phase (b) are visually distinct phases that are packaged in physical contact, wherein the phase (a) and the phase (b) have a yield stress of from about 1 Pa to about 100 Pa, and wherein the viscosity ratio of the phase (a) to the phase (b) is from about 1:15 to about 15:1 at shear stress over the yield stress of the phases (a) and (b) up to at least 200 Pa.

2. The multi-phase personal care composition of claim 1 wherein said viscosity ratio of the phase (a) to the phase (b) is from about 1:5 to about 5:1.

3. The multi-phase personal care composition of claim 1 wherein said viscosity ratio of the phase (a) to the phase (b) is from about 1:3 to about 3:1

4. The multi-phase personal care composition of claim 1 wherein the phase (a) and the phase (b) have a zero shear viscosity of from about 10 kPa·s to about 1,000 kPa·s.

5. The multi-phase personal care composition of claim 1 wherein the phase (a) and the phase (b) have a density of from about 0.85 g/cm³ to about 1.15 g/cm³, and wherein the density difference between the phase (a) to the phase (b) is about 0.20 g/cm³ or less.

6. The multi-phase personal care composition of claim 1 wherein the personal care composition phases (a) and (b) are conditioning composition phases comprising a gel matrix comprising a cationic surfactant, a high melting point fatty compound, and aqueous carrier.

7. The multi-phase personal care composition of claim 6 wherein at least one of the personal care composition phases further contain: a particle having a particle size of from about 5 nm to about 5 μm; and a carrier fluid for the particle having ClogP value of −0.5 or less.

8. The multi-phase personal care composition of claim 7 wherein the particle is selected from the group consisting of organic pigments, inorganic particles, and mixtures thereof.

9. The multi-phase personal care composition of claim 7 wherein the carrier fluid for particle has ClogP value of −1.0 or less.

10. The multi-phase personal care composition of claim 7 wherein the carrier fluid for the particle has ClogP value of −1.5 or less.

11. The multi-phase personal care composition of claim 7 wherein the carrier fluid for particle is selected from the group consisting of diglycerine, sorbitol, glycerin, ethanediol, diethyleneglycol, 1,4-butanediol, propylene glycol, 1,4-pentanediol, 1,3-butanediol, dipropyleneglycol, 1,5-pentanediol, and mixtures thereof.

12. The multi-phase personal care composition of claim 9 wherein the carrier fluid for particle is selected from the group consisting of diglycerine, glycerin, ethanediol, diethyleneglycol, 1,4-butanediol, propylene glycol, and mixtures thereof.

13. The multi-phase personal care composition of claim 10 wherein the carrier fluid for particle is selected from the group consisting of diglycerine, glycerin, and mixtures thereof.

14. The multi-phase personal care composition of claim 7 wherein the personal care composition phase containing the particle and the carrier fluid for particle is substantially free of an oily compound other than the high melting point fatty compound.

15. The multi-phase personal care composition of claim 1 wherein the phase (a) comprising:
(i) a gel matrix comprising a cationic surfactant, a high melting point fatty compound, and an aqueous carrier; and
(ii) a silicone compounds;
wherein the phase (a) is substantially free of a particle having a particle size of from about 5 nm to about 5 μm;
and wherein the phase (b) comprising:
(i) a gel matrix comprising a cationic surfactant, a high melting point fatty compound, and an aqueous carrier;
(ii) a particle having a particle size of from about 5 nm to about 5 μm; and
(iii) a carrier fluid for the particle having a ClogP value of −0.5 or less;
and wherein the phase (b) is substantially free of an oily compound other than the high melting point fatty compounds.

16. The multi-phase personal composition of claim 1 wherein said visually distinct phases form a pattern selected from group consisting of striped, swirl, spiral, marbled, and combinations thereof.

17. The multi-phase personal care composition of claim 1 wherein the personal care composition is a hair conditioning composition.

18. A method of delivering personal care benefits to hair or skin, said method comprising the steps of:
(a) topically applying an effective amount of a composition according to claim 1 onto said hair or skin; and
(b) removing said composition from said hair or skin by rinsing with water.
